(19) United States
(12) Patent Application Publication
(10) Pub. No.: US 2002/0187496 A1
(43) Pub. Date: Dec. 12, 2002

(54) Genetic research systems

(76) Inventors: Leif Andersson, Uppsala (SE); L. Holger Luthman, Bromma (SE); Vidar Wendel-Hansen, Uppsala (SE)

Correspondence Address: MARK S. ELLINGER, Fish & Richardson P.C., P.A., Suite 3300, 60 South Sixth Street, Minneapolis, MN, 55402, US

(21) Appl. No.: 10/086,788

(22) Filed: Feb. 28, 2002

Related U.S. Application Data
(60) Provisional application No. 60/227,342, filed on Aug. 23, 2000.

(30) Foreign Application Priority Data
Aug. 23, 2001 (IB) PCT/IB01/01883

(51) Int. Cl. C12Q 1/68; G06F 19/00

(52) U.S. Cl. 435/6; 702/20

(57) ABSTRACT

The invention relates to systems having flexible genetic information storage, processing, and analysis structures. The disclosed systems can be securely and independently accessed and used multiple researchers and research groups. Such systems can facilitate the collaboration of genetic researchers and research groups.

### CROSS-REFERENCE TO RELATED APPLICATIONS

This application claims priority from Serial No. PCT/U301/41850, filed on Aug. 23, 2001, which claims priority to U.S. Provisional Application Serial No. 60/227,342, filed on Aug. 23, 2000.

### TECHNICAL FIELD

The invention relates to systems useful for storing, processing, and analyzing genetic research data.

### BACKGROUND

Genetic research involves studying inherited traits, often to identify genetic markers associated with particular health problems. Using such genetic markers, clinicians can better predict the likelihood that an individual will develop a particular health problem, or pass on a health risk to their children. Thus, researchers around the world have engaged in intense efforts to identify health-relevant genetic markers.

Genetic research can be time and resource intensive. This is because genetic research efforts often involve collaborations between geographically distributed researchers, and because substantial computing resources and specialized algorithms are required to process and analyze vast amounts of genetic research data.

### SUMMARY

The invention features genetic research systems that can facilitate collaboration between genetic researchers. Genetic research systems in accordance with the invention have flexible structures for storing, processing and analyzing genetic research data provided by different research groups, and can provide secure and independent access to multiple researchers and research groups. Researchers can use a variety of computing devices to access genotype and phenotype data in a genetic research system via a network, interacting with an interface provided by a front-end gateway.

In one aspect the invention relates to genetic research systems that include interrelated data structures to store the following types of data: genotype data ad phenotype data obtained from individuals belonging to different sampling units; phenotype data obtained from individuals belonging to a plurality of sampling units; information about genetic research projects that include one or more of the sampling units; information about biological species that are studied in the genetic research projects; information about the chromosomes of the biological species; information about roles that users may be assigned in the projects; information about the operations that the users can perform using the system; information about the users; information about the sampling units; information about the sampled individuals; information about samples obtained from the individuals; information about genetically relevant groupings to which the individuals can belong; information about genetically relevant groups within the groupings; information about the phenotypic traits measured or observed for individuals in the sampling groups; information about the variables that are to be used when generating data files; information about genetic markers examined for individuals in the sampling groups; information about alleles of one or more of the genetic markers; information about the genetic markers that are to used when generating data files; and information about the genetic position of the markers.

A genetic research system also can include proxy data structures that permit the collective analysis of genotype data and phenotype data linked to particular sampling groups. A proxy data structure for phenotype data can include a data structure to store information about unified variables that refer to and associate variables that pertain to different sampling groups, and a data structure to store information about the unified variables that are to be used when generating data files. A proxy data structure for genotype data can include a data structure to store information about unified markers that refer to and associate markers that pertain to different sampling groups, a data structure to store information about unified alleles that refer to and associate alleles that pertain to different sampling groups, and a data structure to store information about the unified markers that are to be used when generating data files. A proxy data structure for genotype data also can include a data structure to store information about unified positions that refer to and associate positions that pertain to different sampling groups.

In another aspect, the invention provides a method for providing access to a genetic research system. The method involves: a) receiving a request from a user to access a genotype data structure within the system, where the genotype data structure includes nucleic acid sequence data and a level attribute; b) querying a project data object within the system to determine which entries within the genotype data structure the user can access; c) querying a role data structure and a privileges data structure within the system to determine a set of operations that the user is allowed to perform; and d) providing access based on the results of the queries.

In another aspect, the invention provides a method for providing genetic research information to a user. The method involves: a) providing a user access to a genetic research system including one or more genotype data structures to store genotype data obtained from individuals belonging to a plurality of sampling units, and one or more phenotype data structures to store phenotype data obtained from individuals belonging to a plurality of sampling units; b) using one or more genotype proxy data structures to associate genotype data for individuals in different sampling units while maintaining genotype data for individual sampling units in the genotype data structures; c) using one or more phenotype proxy data structures to associate phenotype data for individuals in different sampling units while maintaining phenotype data for individual sampling units in the phenotype data structures; and d) providing the user with information derived from the associated phenotype data and the linked genotype data.

Various embodiments of the invention are set forth in the accompanying drawings and the description below. Other features and advantages of the invention will become apparent from the description, the drawings, and the claims.

Unless otherwise defined, all technical and scientific terms used herein have the meaning commonly understood by one of ordinary skill in the art to which this invention belongs. All publications, patent applications, patents, and other references mentioned herein are incorporated by reference in their entirety. In case of conflict, the present specification, including definitions, will control. The disclosed materials, methods, and examples are illustrative only and not intended to be limiting. Skilled artisans will appreciate that methods and materials similar or equivalent to those described herein can be used to practice the invention.

### DESCRIPTION OF DRAWINGS

FIG. 1 is a block diagram that illustrates a distributed genetic research environment, including a genetic research system in accord with the invention.

FIG. 2 is a block diagram that illustrates in more detail the genetic research system shown in FIG. 1, including a database system in accord with the invention.

FIGS. 3-8 are block diagrams that illustrate in more detail the portions (i.e., “database system modules”) of the database system shown in FIG. 2.

FIGS. 9 and 10 illustrate output that is produced by a researcher using a genetic research system.

FIG. 11 is a block diagram that illustrates in more detail a computer system that a researcher in a genetic research environment can use to interact with a genetic research system.

### DETAILED DESCRIPTION

### Genetic Research Environment and System Configuration

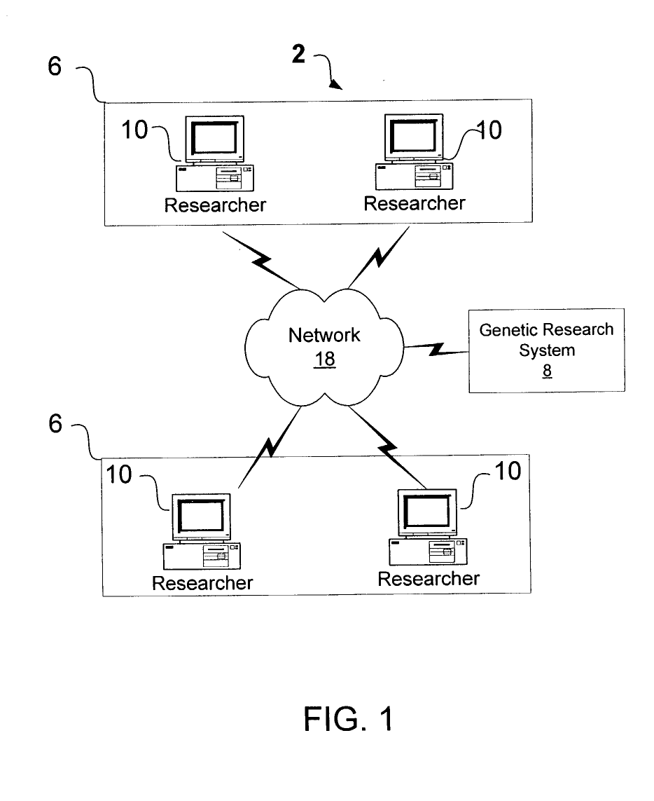

Genetic research systems in accordance with the invention provide flexible information storage, processing, and analysis structures that can facilitate collaboration between genetic researchers in a distributed genetic research environment. Referring to FIG. 1, a distributed genetic research environment 2 has multiple research groups 6, each group including one or more researchers. Within each research group 6, the individual researchers typically collaborate to accomplish a common goal (e.g., to identify genetic markers associated with a particular health condition).

Researchers use a computing device 10 to access a genetic research system 8 via a network 18. Computing device 10 can be any computing device that can interact with network 18 and genetic research system 8. Suitable computing devices include, for example, desktop computers, laptop computers, handheld computers, personal digital assistants (e.g., Palm™ organizers from Palm Inc. of Santa Clara, Calif.), and network-enabled cellular telephones. Network 18 can be any transmission medium suitable for transmitting digital data. For example, network 18 can be a packet-based digital network, such as a private wide area network (WAN) or the Internet, running a network protocol, such as the transmission control protocol/internet protocol (TCP/IP). A communication tool, such as a web browser like Internet Explorer™ from Microsoft Corporation of Redmond, Wash., executes in an operating environment on computing device 10 and allows a researcher to access genetic research system 8.

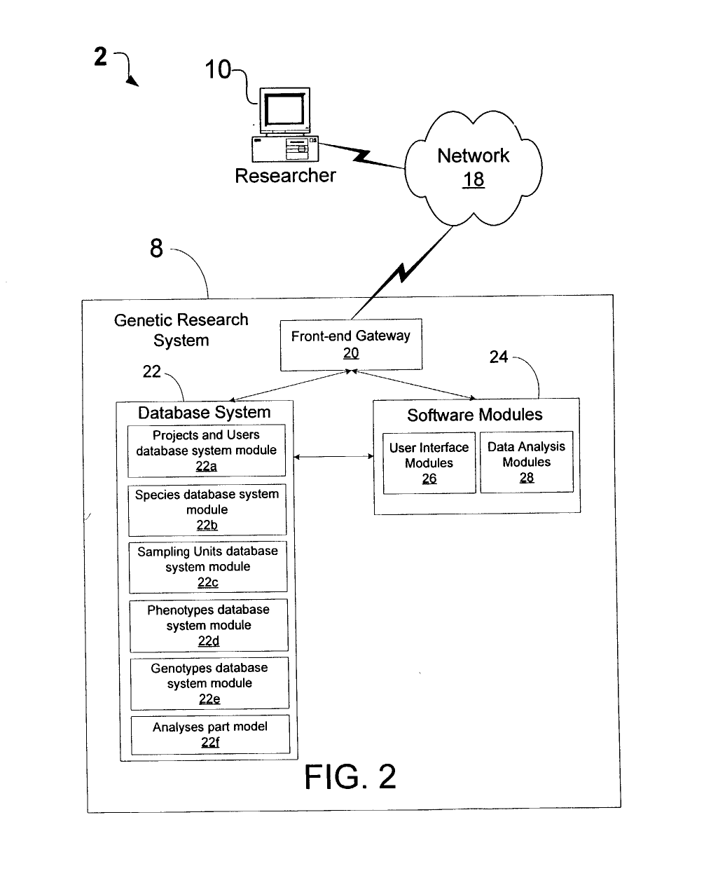

Referring to FIG. 2, genetic research system 8 includes three components: 1) at least one front-end gateway 20, 2) software modules 24, and 3) a database system 22 for storing and processing genetic research data. Front-end gateway 20 (e.g., a web server) provides a communication interface that mediates the interaction of computing device 10 with genetic research system 8 via network 18. Thus, front-end gateway 20 typically executes server software, such as Internet Information Server™ (Microsoft Corp.), or Apache Web Server™ software. A front-end gateway 20 can be implemented on the same machine as a database system 22. Alternatively, front-end gateway 20 can be communicatively coupled to database system 22 that is implemented on a database server using a database engine, such as Oracle™. In such a configuration, front-end gateway 20 and a database server that implements database system 22 typically are linked via a packet-based local area network (LAN),

Communication between computing device 10 and front-end gateway 20 can be encrypted. Thus, front-end gateway 20 can require computing device 10 to use an HTTPS (i.e., HTTP plus SSL) protocol, and participate in a reciprocal certificate authentication process. Authentication certificates for computing device 10 and front-end gateway 20 can be generated by a certificate authority, and can be distributed to computing device 10 by, for example, removable media. Communication between computing device 10 and front-end gateway 20 also can require a password. Thus, front-end gateway 20 can require computing device 10 to provide a valid username and password before allowing access to database system 22 or to software modules 24. Usernames and passwords can be sent to front-end gateway 20 in encrypted form (e.g., after a certificate authentication process). Communication between computing device 10 and front-end gateway 20 can be time-limited. Thus, front-end gateway 20 can use cookies to measure time intervals (e.g., after login, or between communications) during an active session with computing device 10. Front-end gateway 20 can terminate an active session after a predefined time interval.

Software modules 24 of genetic research system 8 include user interface modules 26 and data analysis modules 28. User interface modules 26 include program instructions to provide interface forms from which a user can store, access, edit, and analyze genetic research data in database system 22. Data analysis modules 28 include program instructions for analyzing genetic research data stored in a database system 22 (e.g., to locate and map multiple interacting quantitative trait loci (QTL) in a genome). Program instructions in software modules 24 can include, for example, Lotus scripts, Java scripts, Java Applets, Java servlets, Active Server Pages, web pages written in hypertext markup language (HTML) or dynamic HTML, Active X modules, CGI scripts, and other suitable modules such as stand-alone executables written in C or C++. Such program instructions also can be called by software modules 24 from database system 22.

### Database System Information Structure

The information structure of database system 22 can be described in terms of interrelated portions, or “database system modules.” In the implementation shown in FIG. 2, database system 22 includes the following database system modules: 1) a Projects and Users database system module 22a, 2) a Species database system module 22b, 3) a Sampling Units database system module 22c, 4) a Phenotypes database system module 22d, 5) a Genotypes database system module 22e, and 6) an Analyses database system module 22f. Each database system module is described in detail below.

By way of general introduction, a database system module includes database objects and relationships between database objects. Database objects define data structures for storing and organizing data in a database, and relationships between database objects define whether and how information stored in database objects is associated. In graphical database schema, database objects are represented by rectangular boxes and relationships between database objects are represented by lines and their end points. Dashed lines in database schema indicate relations that may or may not be fulfilled. A line having one large endpoint indicates a one-to-many relationship between the database objects that it connects, and a line having two large endpoints indicates a many-to-many relationship between the database objects that it connects. Smaller filled squares at junction points between lines indicate relations between more than two objects.

Database objects can be dynamic. That is, the entries included in database objects can change over time as data is added, deleted, or otherwise modified. A history of changes for a database object can be monitored and recorded (e.g., in a linked history object).

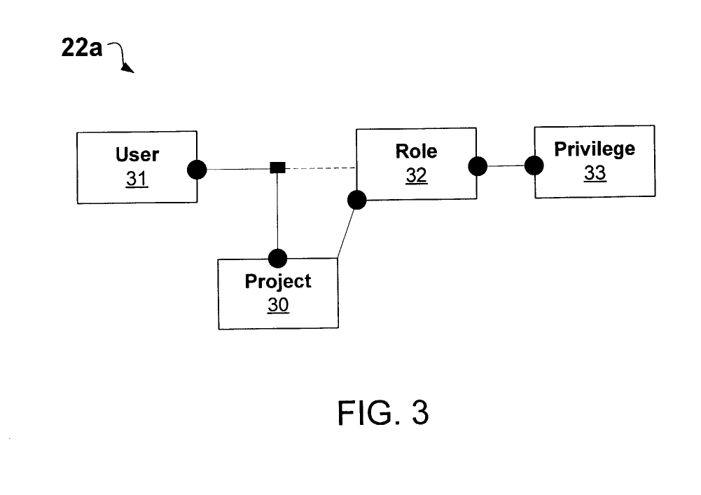

Projects and Users database system module: In general, a Projects and Users database system module 22a, an example of which is shown in FIG. 3, dictates which researchers can participate in particular genetic research projects (e.g., projects aimed to identify genetic markers associated with particular health conditions). By way of illustration, Projects and Users database system module 22a can dictate that Researcher A has access to a hypertension marker project, that Researcher B has access to a tumor marker project, and that Researcher C has access to a stroke marker project. A Projects and Users database system module 22a also dictates what functions particular researchers can perform with respect to particular genetic research projects. By way of illustration, a Projects and Users database system module 22a can dictate that Researcher A has display access, that Researcher B has display and edit access, and that Researcher C has display, edit and analyze access.

The Projects and Users database system module shown in FIG. 3 includes a three-way relationship between User object 31, Project object 30 and Role object 32, one part of which may or may not be fulfilled. The module also includes a one-way relationship between Role object 32 and Project object 30. In this configuration, a project has one or more roles associated with it (one-way, one to many relationship between project and role), and a user may or may not have a role in the project (dashed line). In this configuration, a user can have only one role in a particular project, but can have different roles in different projects. In this configuration, more that one project member can have the same role.

A Role object 32 and a Privileges object 33 define the operations that a user can perform using genetic research system 8. An entry in Role object 32 can map to one or multiple entries in Privileges object 33, and an entry in Privileges object 33 can map to one or multiple entries in Role object 32. This configuration defines a research system in which a particular role can be assigned more than one privilege, and in which a particular privilege can be assigned to more than one role. The role of project administrator typically is assigned to at least one project member. A project administrator typically can create and edit project roles, add and remove project members, and reassign roles for project members. A system administrator typically can define users' access to projects, create, edit and delete Project object entries, and create, edit and delete User object entries. Typically, only a system administrator can create User objects and Project objects.

Table 1 lists exemplary objects, including attributes for stored entries, which can be included in a Projects and Users database system module.

TABLE 1

| | | |
|---|---|---|
| Attribute | Type | Description |

Project object

| | | |
|---|---|---|
| Name | Text | Project name (unique within system). |
| Comment | Text | Project description. |
| Status | Text | Project status (enabled or disabled). Users can login to |
| | | enabled projects. |

User object

| | | |
|---|---|---|
| Identity | Text | Login identity for user (i.e., username) (unique within |
| | | system). |
| Password | Text | User password |
| Name | Text | Name of user. |
| Status | Text | Status for user (enabled or disabled). Enabled users |
| | | can login to system. |

Role object

| | | |
|---|---|---|
| Name | Text | Name of role, e.g. project leader (unique within |
| | | project). |
| Comment | Text | Role description. |

Privileges object

| | | |
|---|---|---|
| Name | Text | Short name of privilege. |
| Comment | Text | Privilege description. |

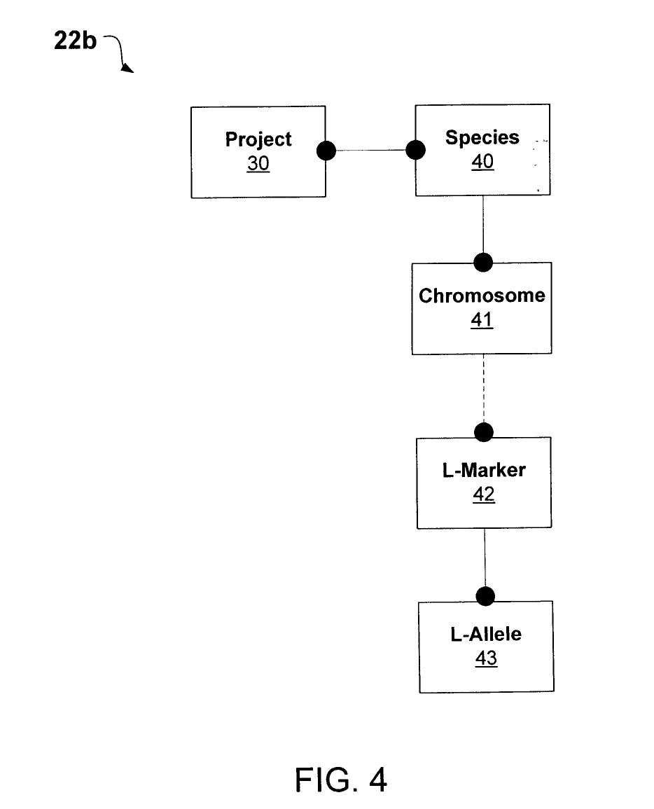

Species database system module: In general, a Species database system module 22b, an example of which is shown in FIG. 4, models biological species and their relevant genetic features. Biological species include, for example Homo sapiens, Pan troglodytes, and Rattus norvegicus. The Species database system module shown in FIG. 4 includes a Species object 40 that can contain information about a biological species, including its name. An entry in Species object 40 can relate to one or more entries in Project object 30, and an entry in Project object 30 can relate to one a single entry in Species object 40. Thus, a species can be included in one or more research projects, each of which relates to a single species.

One genetic feature of a biological species is its chromosome(s). Humans, for example, have 46 chromosomes and 24 chromosome types (i.e., 1, 2, . . . 22, X, and Y). Other genetic features of biological species include genetic markers and alleles. Genetic markers, or markers, refer to genetic loci on a chromosome, the nucleic acid sequence of which can be polymorphic among the members of a biological species. Nucleic acid sequence variants of particular genetic markers are called alleles. Referring again to FIG. 4, entries in a Chromosome object 41 contain information about particular chromosomes, including their names. Since biological species can have multiple chromosomes, an entry in Species object 40 can relate to one or more entries in Chromosome object 41. An L-marker object 42 can include information about markers, such as their genetic location on a chromosome, nucleic acid primers that can be used to obtain nucleic acid copies of the markers (e.g., by the polymerase chain reaction), or to determine the nucleic acid sequence at the markers in particular individuals. An L-allele object 43 can include information about marker alleles. Since chromosomes can have multiple genetic markers and since genetic markers can have multiple alleles, an entry in Chromosome object 41 can relate to one or more entries in L-marker object 42, and an entry in L-marker object 42 can relate to one or more entries in L-allele object 43. Species, Chromosome, L-marker and L-allele objects typically are created by a system administrator.

Table 2 lists exemplary objects, including attributes for stored entries, which can be included in a Species database system module.

TABLE 2

| | | |
|---|---|---|
| Attribute | Type | Description |

Species object

| | | |
|---|---|---|
| Name | Text | Name of species, e.g. human (unique within sys- |
| | | tem). |
| Comment | Text | Species description. |

Chromosome object

| | | |
|---|---|---|
| Name | Text | Name of chromosome, e.g. “22” or “X” (unique |
| | | within species). |
| Comment | Text | Chromosome description. |

L-Marker object

| | | |
|---|---|---|
| Name | Text | Marker name (unique within species). |
| Alias | Text | Marker alias. |
| Position | Number | Genetic chromosome position for marker (can be |
| | | null). |
| Primer1 | Text | Primer 1 (can be null). |
| Primer2 | Text | Primer 2 (can be null). |
| Comment | Text | Marker description |

L-Allele object

| | | |
|---|---|---|
| Name | Text | Allele name or identity (unique within library |
| | | marker). |
| Comment | Text | Allele description. |

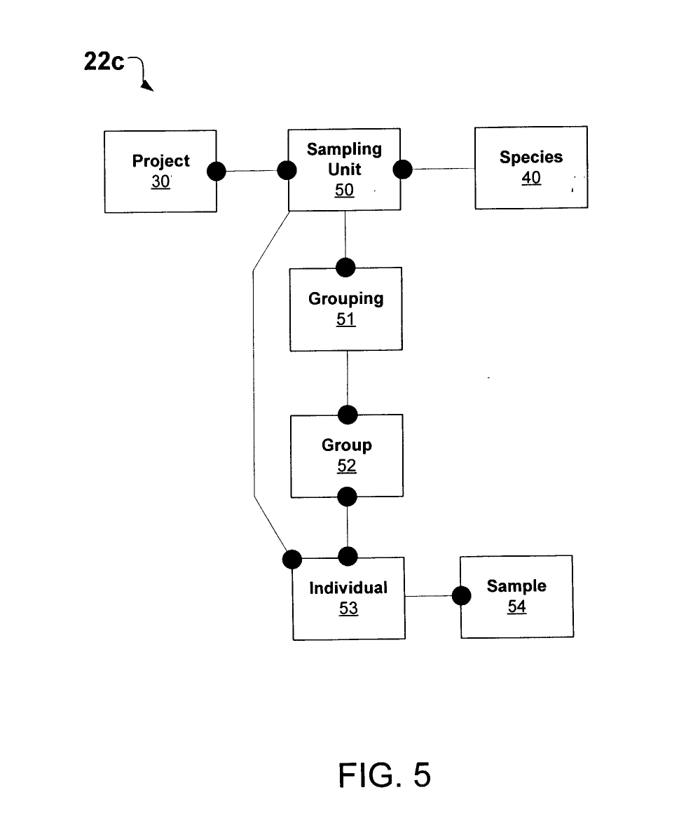

Sampling Units database system module: In general, a Sampling Units database system module 22c, an example of which is shown in FIG. 5, organizes information about individuals from whom samples have been obtained. A sampling unit can include one or more individuals from whom samples have been obtained. For example, a sampling unit can include individuals sampled by a particular research group, at a particular place, or at a particular time. The Sampling Units database system module shown in FIG. 5 includes a Sampling Unit object 50 that can contain information about sampling units, including names and descriptions. A sampling unit can include one or more individuals. Thus an entry in Sampling Unit object 50 can relate to one or more entries in an Individual object 53, which can contain information about individuals. A project can involve one or more sampling units, and a sampling unit can be used by one or more projects. Thus, an entry in Project object 30 can relate to one or more entries in Sampling Unit object 50, and an entry in Sampling Unit object 50 can relate to one or more entries in Project object 30. Such a configuration allows different sub-populations of sampled individuals to be considered in particular research projects; a genetic research analysis need not collectively consider all individuals, and particular research projects can consider different sub-populations of sampled individuals. This is one manner that genetic research system 8 can facilitate the collaboration between genetic researchers in a distributed genetic research environment. Genetic researchers in different research groups can share information obtained from sampled individuals, and particular research groups can select particular sampling units for analysis.

Entries in a Sample object 54 can store information about samples, including the type of sample, date it was obtained, and manner in which it was preserved. Multiple samples can be obtained from an individual. Thus, an entry in Individual object 53 can relate to one or more entries in Sample object 54. Individuals included in a sampling unit can belong to various genetically relevant groupings (e.g., generation and family), and to groups within groupings (e.g., a particular family or a particular generation). A Grouping object 51 can store information about genetically relevant groupings, and a Group object 52 can store information about genetically relevant groups within groupings. Since an individual can belong to more than one genetically relevant group, an entry in Individual object 53 can relate to one or more entries in Group object 52. Since a grouping belongs to particular group, an entry in Group object 52 relates to one entry in Grouping object 51.

Table 3 lists exemplary objects, including attributes for stored entries, which can be included in a Sampling Units database system module.

TABLE 3

| | | |
|---|---|---|
| Attribute | Type | Description |

Sampling Unit object

| | | |
|---|---|---|
| Name | Text | Sampling unit name (unique within system). |
| Comment | Text | Sampling unit description. |
| Status | Text | Status for sampling unit (enabled or disabled). Projects can work |
| | | with enabled sampling units. |

Individual object

| | | |
|---|---|---|
| Identity | Text | Individual name (unique within sampling unit). |
| Alias | Text | Alias for individual (unique within sampling unit; can be null). |
| Father | Reference | Reference to father (can be null). |
| Mother | Reference | Reference to mother (can be null) |
| Sex | Text | Male, female or unknown. |
| Birth date | Date | Date of birth (can be null). |
| Comment | Text | Individual description. |
| Status | Text | Status for individual (enabled or disabled). Disabled individuals are |
| | | treated as non-existent when data files are generated. |

Grouping object

| | | |
|---|---|---|
| Name | Text | Grouping name. |
| Comment | Text | Grouping description. |

Group object

| | | |
|---|---|---|
| Name | Text | Group name. |
| Comment | Text | Group description. |

Sample object

| | | |
|---|---|---|
| Name | Text | Sample name (unique within individual). |
| Tissue | Text | Tissue type (can be null). |
| Experimenter | Text | Name of experimenter (can be null). |
| Date | Date | Date of sample (can be null). |
| Treatment | Text | Sample treatment (can be null). |
| Storage | Text | Sample storage, e.g. “frozen” (can be null). |
| Comment | Text | Sample comment. |

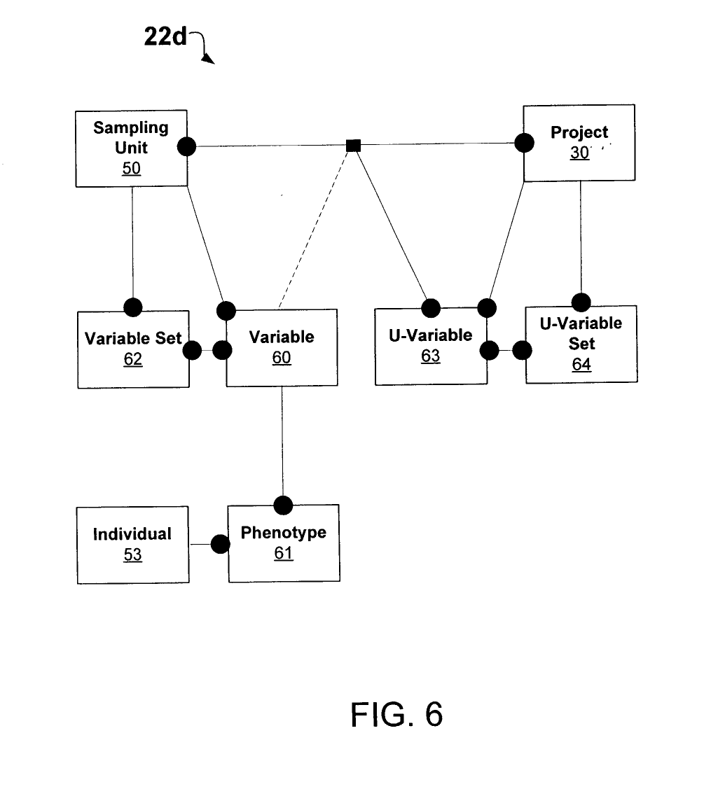

Phenotypes database system module: In general, a Phenotypes database system module 22d, an example of which is shown in FIG. 6, organizes and facilitates the analysis of information related to variables that have been determined for sampled individuals. A variable is a trait that can be observed or measured (e.g., by physical or biochemical analysis), including, for example, physical traits, mental traits, physiological traits, neurological traits, and behavioral traits. A phenotype is the actual value or observation recorded for such traits. The species module shown in FIG. 6 includes a Phenotype object 61 that can contain information about observations or measurements made for sampled individuals. Since phenotypes can be observed or measured one or more times for a particular individual, an entry in Individual object 53 can relate to one or more entries in Phenotype object 61.

A Variable object 60 and a Variable Set object 62 dictate which variables and phenotypes are included when generating data files for analyses that involve a single sampling unit. Variable object 60 can include information about traits that are measured or observed for individuals in a sampling unit. Since a variable can be observed or measured (i.e., as a phenotype) one or more times for one or more individuals, an entry in Variable object 60 can relate to one or more entries in Phenotype object 61. Variable Set object 62 can include information about which variables are to be included when generating data files. A variable set can include multiple variables, and a variable can be included in multiple variable sets. Thus, an entry in Variable Set object 62 can relate to one or more entries in Variable object 60, and an entry in Variable object 60 can relate to one or more entries in Variable Set object 62.

A Unified Variable (U-variable) object 63 and a Unified Variable Set (U-variable set) object 64 dictate which variables are included when generating data files for analyses involving multiple sampling units. U-variable object 63 can include information about traits that are measured or observed for individuals that belong to different sampling units. An entry in U-variable object 63 (i.e., a unified variable) can be used to refer to and associate variables for a variety of different sampling units. Thus, an entry in Variable object 60 can relate to one or more entries in U-variable object 63. U-variable Set object 64 can include information about which unified variables are to be included when generating data files. A unified variable set can include multiple unified variables, and a unified variable can be included in multiple unified variable sets. Thus, an entry in U-variable Set object 64 can relate to one or more entries in U-variable object 63, and an entry in U-variable object 63 can relate to one or more entries in U-variable Set object 64.

By way of illustration, consider a project that involves two sampling units, S1 and S2. Information about S1 and S2 is included in separate entries in Sampling Unit object 50. Each sampling unit has its own variables for weight, WT for S1 and WGT for S2. Information about WT and WGT is included in separate entries in Variable object 60, and measured values for WT and WGT are included in separate entries in Phenotype object 61. A unified variable called UWEIGHT can be used to treat the variables WT and WGT as the same variable, and thereby allow the same type of phenotype data (i.e., weight) for individuals belonging to different sampling units to be analyzed together.

This is another example of how a genetic research system can facilitate the collaboration between genetic researchers in a distributed genetic research environment. Implementing separate but related database objects for non-unified variables and corresponding unified variables (i.e., proxy data structures) permits the collective analysis of phenotype data from multiple sampling units, and discrete analysis of phenotype data from individual sampling units. Genetic researchers in different research groups can share and pool phenotype information obtained from sampled individuals while information regarding individual sampling units is maintained for discrete analysis.

Table 4 illustrates exemplary objects, including attributes for stored entries, which can be included in a Phenotypes database system module.

TABLE 4

| | | |
|---|---|---|
| Attribute | Type | Description |

Variable object

| | | |
|---|---|---|
| Name | Text | Variable name, e.g. “weight” (unique within sampling |
| | | unit). |
| Type | Text | Variable type (enumeration or number). |
| Unit | Text | Measuring unit, e.g. “kg” or “cm.” |
| Comment | Text | Variable description. |

Variable Set object

| | | |
|---|---|---|
| Name | Text | Variable set name (unique within sampling unit). |
| Comment | Text | Variable set name. |

Phenotype object

| | | |
|---|---|---|
| Value | Text | Observed value. |
| Date | Date | Date of observation (can be null). |
| Reference | Text | Reference to raw data for observation (can be null). |
| Comment | Text | Phenotype comment. |

U-Variable object

| | | |
|---|---|---|
| Name | Text | Unified variable name, e.g. “weight” (unique within |
| | | project and species). |
| Comment | Text | Unified variable description. |

U-Variable Set object

| | | |
|---|---|---|
| Name | Text | Unified variable set name (unique within project). |
| Comment | Text | Unified variable set name. |

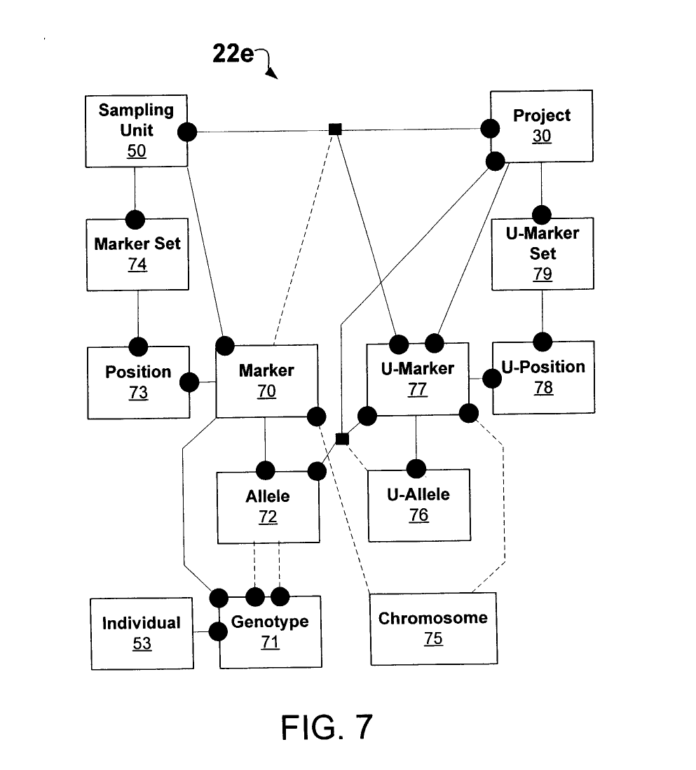

Genotypes database system module: In general, a Genotypes database system module 22e, an example of which is shown in FIG. 7, organizes and facilitates the analysis of genetic information obtained from sampled individuals. Genetic information includes information about genetic markers. Genetic markers, or markers, refer to genetic loci on a chromosome, the nucleic acid sequence of which can be polymorphic among the members of a biological species. Nucleic acid sequence variants of particular genetic markers are called alleles. The species module shown in FIG. 7 includes a Genotype object 71 that can contain information about nucleic acid sequence data determined for sampled individuals. Multiple nucleic acid sequence determinations can be made for a particular individual (e.g., for different markers, or for the two alleles of a marker in biological species that have pairs of like chromosomes). Thus, an entry in Individual object 53 can relate to one or more entries in Genotype object 71. To preserve the integrity of raw genetic research data, an entry in Genotype object 71 can store a level attribute that defines the security level of entries in Genotype object 71. Project members can have different privileges corresponding to different security levels. For example, a project member having privilege level five can access create or update genotype data having level five or less, and a project leader having level nine privileges can lock genotype data by setting the level to nine.

A Marker object 70 and an Allele object 72 can include information about markers and alleles examined for individuals in a sampling unit, respectively. Since an allele can be observed in more than one individual, an entry in Allele object 72 can relate to one or more entries in Genotype object 71. Since a marker can have multiple alleles, a single entry in Marker object 70 can relate to one or more entries in Allele object 72. Marker object 70 also can include position information useful for calculating genetic distances between markers. A Position object 73 also can include a value used for ordering or calculating distances between markers positioned on the same chromosome.

A Marker Set object 74 dictates which markers are to be included when generating data files for analyses that involve a single sampling unit. The relationship between marker sets and markers can be implemented by Position object 73 such that an entry in Marker Set object 74 relates to one or more entries in Position object 73, each of which relates to an entry in Marker object 70. Thus, a marker set defines a set of positions, each of which references a marker that is to be included when generating data files.

A Unified Marker (U-marker) object 77, a Unified Marker set (U-marker set) object 79, a Unified Allele (U-allele) object 76 and a Unified Position (U-position) object 78 dictate which markers are included when generating data files for analyses involving multiple sampling units. U-marker object 77 can include information about markers that are examined for individuals in different sampling units. An entry in U-marker object 77 (i.e., a unified marker) can be used to refer to and associate markers for a variety of different sampling units. Thus, an entry in Marker object 70 can relate to one or more entries in U-marker object 77. U-allele object 76 can include information about alleles that are examined for individuals in different sampling units. An entry in U-allele object 76 (i.e., a unified allele) can be used to refer to and associate alleles for a variety of different sampling units. Thus, an entry in U-allele object 76 can relate to one or more entries in Allele object 72. A U-marker set object 79 can include information about which unified markers are to be included when generating data files. The relationship between unified marker sets and unified markers can be implemented by U-position object 78 such that an entry in U-marker Set object 79 relates to one or more entries in U-position object 78, each of which relates to one entry in U-marker object 77. Thus, a unified marker set defines a set of U-positions, each of which references a marker that is to be included when generating data files. U-marker object 77 and U-position object 78 also can include position information useful for calculating genetic distances between markers.

This is another example of how a genetic research system can facilitate the collaboration between genetic researchers in a distributed genetic research environment. Implementing separate but related database objects for non-unified and corresponding unified markers and alleles (i.e., proxy data structures) permits the analysis of genotype data from individual sampling units, and the collective analysis of genotype data from a variety of different sets of sampling units. Genetic researchers in different research groups can share and pool genotype information obtained from sampled individuals while information regarding particular sampling units is maintained for discrete analysis.

Table 5 illustrates exemplary objects, including attributes for stored entries, which can be included in a Genotypes database system module.

TABLE 5

| | | |
|---|---|---|
| Attribute | Type | Description |

Marker object

| | | |
|---|---|---|
| Name | Text | Marker name (unique within sampling unit). |
| Alias | Text | Marker alias (unique within sampling unit). |
| Position | Number | Genetic chromosome position for marker (can be |
| | | null). |
| Primer1 | Text | Primer 1 (can be null). |
| Primer2 | Text | Primer 2 (can be null). |
| Comment | Text | Marker description. |

Allele object

| | | |
|---|---|---|
| Name | Text | Allele name (unique within marker). |
| Comment | Text | Allele description. |

Genotype object

| | | |
|---|---|---|
| Raw data 1 | Text | Raw data value for allele 1. |
| Raw data 2 | Text | Raw data value for allele 2 (can be null). |
| Reference | Text | Reference to raw data, e.g. “microfilm” or “gel.” |
| Comment | Text | Comment. |
| Level | Integer | Confidence or security level. |

Marker Set object

| | | |
|---|---|---|
| Name | Text | Marker set name (unique within sampling unit). |
| Comment | Text | Marker set description. |

Position object

| | | |
|---|---|---|
| Value | Number | Genetic position for marker (in cM, can be null). |

U-Marker object

| | | |
|---|---|---|
| Name | Text | Unified marker name (unique within project). |
| Alias | Text | Unified marker alias (unique within project). |
| Position | Number | Genetic chromosome position for marker (can be |
| | | null). |
| Comment | Text | Unified marker description. |

U-Allele object

| | | |
|---|---|---|
| Name | Text | Unified allele name (unique within unified |
| | | marker). |
| Comment | Text | Unified allele description. |

U-Marker Set object

| | | |
|---|---|---|
| Name | Text | Unified marker set name (unique within pro- |
| | | ject and species). |
| Comment | Text | Unified marker set description. |

U-Position object

| | | |
|---|---|---|
| Value | Number | Genetic position for unified marker in unified |
| | | marker set (in cM). |

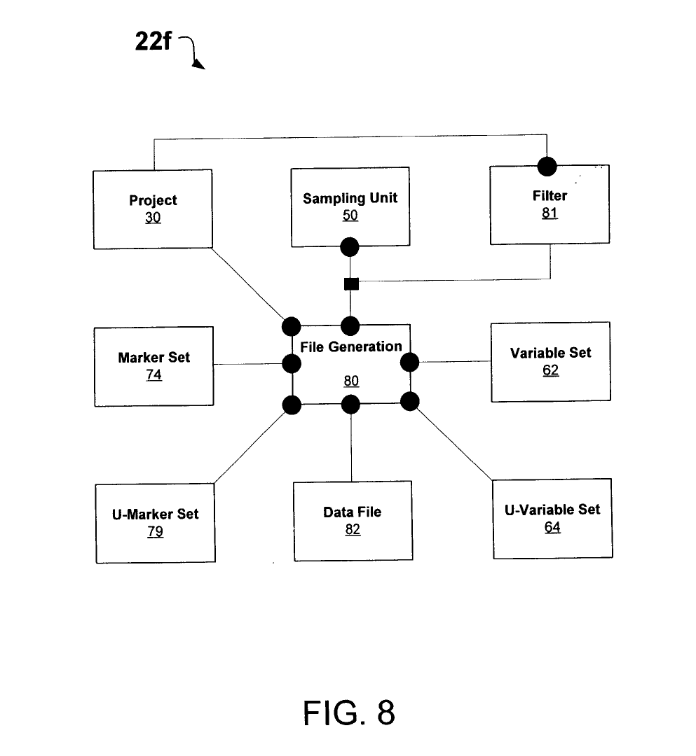

Analyses database system module: In general, an Analyses database system module 22f, an example of which is shown in FIG. 8, can be used to facilitate the analysis of genetic research data. An entry in a File Generation object 80 refers to a set of data files, and relates to one project (i.e., to a single entry in a Project object 30) and to one or more sampling units (i.e., entries in a sampling unit 50). As described above, for file generations involving a single sampling unit, retrieval of phenotype and genotype data for a data file can be determined by a variable set and a marker set. For file generations involving multiple sampling units, retrieval of phenotype and genotype data for a data file can be determined by a unified variable set and a unified marker set.

Filters can be used to select which individuals' data are to be used when generating a data file. A Filter object 35 includes one or more filters, which can be logical, Boolean expressions used for selection of individuals. During the selection process, the expression is evaluated for each individual in a sampling unit. The individuals for which the expression evaluates to true are selected for inclusion when generating a data file. Filter expressions can be written using, for example, a Genetic Query Language (GQL), a simplified syntax that enables scientists lacking detailed knowledge of Structured Query Language (SQL) to write complex queries that can be used as filters for generating analysis files. GQL queries can include standard Oracle™ expressions as well as specialized functions and terms. Thus, GQL expressions can include combinations of parentheses, logical and numerical operators, standard functions and user defined functions. A GQL expression also can include any of the following specialized terms: individual attributes (e.g., sex or birth date), genotype attributes (e.g., allele or raw data for allele), phenotype attributes (e.g., value or date), and set membership (e.g., grouping or group). Individual attributes can be referenced with the prefix “I” (e.g., I.SEX). Genotype attributes can be referenced with the prefix “G” (e.g., G.MA001.A1 for allele 1 of marker MA001). Phenotype attributes can be referenced with the prefix “P” (e.g., P.EYECOLOR). Set membership attributes can be referenced with the prefix S (e.g., S.GENERATIONS for a member of the grouping GENERATIONS, and S.GENERATIONS.F2 for a member of group F2 in the grouping GENERATIONS). The foregoing expressions relate to attributes or membership of an individual under evaluation. Attributes or set membership of an individual's parents or ancestors can be referenced by writing a sequence of M (for mother) or F (for father) after the first prefix.

Thus, P.FM.EYECOLOR.VALUE refers to a value of eye color for an individual's paternal grandmother, and P.MM.EYECOLOR.VALUE refers to a value of eye color for an individual's maternal grandmother.

Table 6 illustrates exemplary objects, including attributes for stored entries, which can be included in an Analyses database system module.

TABLE 6

| | | |
|---|---|---|
| Attribute | Type | Description |

File Generation object

| | | |
|---|---|---|
| Name | Text | File generation name (unique within project). |
| Mode | Text | General mode (single or multiple sampling units). |
| Type | Text | File generation type, e.g. “linkage.” |
| Comment | Text | File generation description. |

Data File object

| | | |
|---|---|---|
| Name | Text | Data file name. |
| Type | Text | Data file type, e.g. “linkage.” |
| Status | Text | Data file status, e.g. “% currently generated.” |
| Comment | Text | Data file description. |

Filter object

| | | |
|---|---|---|
| Name | Text | Filter name, e.g. “males.” |
| Expression | Text | Logical expression (written in GQL). |
| Comment | Text | Filter description. |

### Genetic Research System Interface

To access genetic research system 8, a user typically provides a username and a password. A user that provides a valid username and password can access various interface forms to store, access, process and analyze genetic research data. Interface forms implement the functionality of genetic research system 8, and access to particular forms is governed by a user's roles and associated privileges. Table 7 lists exemplary privileges that allow access to particular interface forms, and thereby functions, of genetic research system 8. Other privileges (e.g., that provide access to different genetic research system functions) can be defined and implemented as a matter of routine by one of skill in the art.

TABLE 7

| | |
|---|---|
| Privilege | Accessible functions |

General privileges

| | |
|---|---|
| PROJ_ADM | Add and delete project members. Add, delete and update project roles. |
| PROJ_STA | View project statistics |

Sampling Unit privileges

| | |
|---|---|
| SU_W | Create, update and delete sampling units. Check sampling units. |
| SU_R | View sampling units |
| GRP_W | Create, copy, update and delete groupings and groups. Edit group membership |
| GRP_R | View groupings, groups and group membership |
| IND_W | Create, update and delete individuals and samples. |
| IND_R | View individuals and samples. |

Phenotype privileges

| | |
|---|---|
| VAR_W | Create, update and delete variables. |
| VAR_R | View variables. |
| VARS_W | Create, update and delete variable sets. Edit variable set membership. |
| VARS_R | View variable sets and variable set membership |
| UVAR_W | Create, update and delete unified variables. Map unified variables. |
| UVAR_R | View unified variables. |
| UVARS_W | Create, update and delete unified variable sets. Edit unified variable set |
| | membership. |
| UVARS_R | View unified variable sets. View unified variable set membership. |
| PHENO_W | Create, update and delete phenotypes. |
| PHENO_R | View phenotypes. |

Genotype privileges

| | |
|---|---|
| MRK_W | Create, update and delete markers and alleles. |
| MRK_R | View markers and alleles. |
| LMRK_R | View and copy library markers and alleles. |
| MRKS_W | Create, update and delete marker sets. Edit marker set membership and positions. |
| MRKS_R | View marker sets, marker set membership and positions. |
| UMRK_W | Create, update and delete unified markers and alleles. Map unified markers and |
| | alleles. |
| UMRK_R | View unified markers and alleles. |
| UMRKS_W | Create, update and delete unified variable sets. Edit unified variable set member- |
| | ship and unified positions. |
| UMRKS_R | View unified variable sets, unified variable set membership and unified positions. |
| GENO_W0 | Create, update and delete genotypes with level = 0. |
| GENO_W1 | Create, update and delete genotypes with level <= 1. |
| GENO_W2 | Create, update and delete genotypes with level <= 2. |
| GENO_W3 | Create, update and delete genotypes with level <= 3. |
| GENO_W4 | Create, update and delete genotypes with level <= 4. |
| GENO_W5 | Create, update and delete genotypes with level <= 5. |
| GENO_W6 | Create, update and delete genotypes with level <= 6. |
| GENO_W7 | Create, update and delete genotypes with level <= 7. |
| GENO_W8 | Create, update and delete genotypes with level <= 8. |
| GENO_W9 | Create, update and delete genotypes with level <= 9. |
| GENO_R | View genotypes. |

Analysis privileges

| | |
|---|---|
| FLT_W | Create, update and delete filters. |
| FLT_R | View filters. |
| ANA_W | Create, update and delete file generations. |
| ANA_R | View file generations and data files. Download data files. |

Provided below are exemplary interface forms, grouped into categories corresponding to the database system modules of database system 22. Other interface forms (e.g., that provide access to different genetic research system functions, or that allow access to users having different privileges) can be designed and implemented as a matter of routine by one of skill in the art.

Projects and Users administration forms: A “set project” form typically is displayed after login, prompting a user to select a project on which to work before allowing access to other interface forms. A user can select a project for which he or she has been assigned a role. System administrators have system-wide privileges and need not select a particular project before using other interface forms. In some configurations, a user can change projects without a separate login event. A user can use a “session options” form to set parameters that control how a system interface behaves during a session (e.g., how null or missing values are displayed, how many rows are displayed in forms, and how dates are formatted).

A project administrator can use a “project members” form to list members of a project, including username, name, role, and status. A project members form also can be used to create project members (i.e., to assign roles to users), update project members' roles, and delete project members. A project administrator can use a “list roles” form to list roles that are linked to particular privileges, including the name of the roles and any associated comments. A “list roles” form also can be used to create roles, update roles (including privilege sets), and delete roles. A project administrator can use an “import role” form to import a role, including its privilege set, from a file. A “project statistics” form can be used to display statistics related to a particular project, including the number of users, number of sampling units, number of individuals, number of variables, number of phenotypes, number of markers, and number of genotypes. Project statistics privileges typically are required to use the form.

A system administrator can use an “edit projects” form to list projects that match one or more of the following search fields: name (search pattern with wildcards), species (choice of one or more), sampling unit (choice of one or more), user (choice of one or more), and status (choice of enabled or disabled). Project names and any associated comments can be displayed. An edit projects form also can be used to create and update projects, link and unlink species to projects, link and unlink sampling units to projects, link and unlink users to projects, create, update and delete roles, and import roles from a file. A system administrator can use a “system statistics” form to obtain project overviews, including information regarding the number of users, number of species, and number of sampling units. A system administrator can use a “list users” form to list users that match one or more of the following search fields: username (search pattern with wildcards) and name (search pattern with wildcards). The names, usernames, and passwords of users can be displayed. A list users form also can be used to create users, update users, and delete users.

Species administration forms: A system administrator can use a “list species” form to list species in a system, including species names, associated comments, and update dates. A list species form also can be used to create species, update species, delete species, view species details (including chromosomes and chromosome details), create chromosomes, update chromosomes, delete chromosomes, and import chromosomes from a file. A system administrator can use a “list L-markers” form to list library markers that match one or more of the following search fields: species, chromosome (choice of one or more), and name (search pattern with wildcards). L-marker names, associated comments, the chromosomes on which L-markers are located, and update dates can be displayed. A list L-markers form also can be used to view details for library markers (including library alleles and library allele details), create library markers, update library markers, delete library markers, create library alleles, update library alleles, and delete library alleles. A system administrator can use an “import L-markers” form to import markers, including alleles, from a file. A system administrator can use an “import project markers” form to import markers from projects.

Sampling Unit administration forms: A user can access a “list sampling units” to list sampling units that are linked to a particular species or that have a particular status. Sampling unit names, associated comments, number of individuals in a sampling unit, updating users, and update dates can be displayed. A list sampling units form also can be used to view sampling unit details, create sampling units, update sampling units, delete sampling units (i.e., unlink from project), and check a sampling unit for errors (e.g., non-existent parent, incorrect parent sex, and incorrect parent birth date).

A user can access a “list groupings” form to list groupings that are linked to a particular sampling unit. Grouping names, associated comments, number of groups, updating users, and update dates can be displayed. A list groupings form also can be used to view grouping details, create groupings, update groupings, delete groupings, and copy groupings (i.e., copy groups to a new grouping). A user can access an “import groupings” form to import new groupings, including groups and group members, from a file.

A user can access a “list groups” form to list groups that are linked to a particular sampling unit and/or grouping. Group names, associated comments, number of individuals, updating users, and update dates can be displayed. A list groups form also can be used to view group details, create groups, update groups, delete groups, and copy groups to a different grouping. A user can access a “group membership” form to add or delete group members.

A user can access a “list individuals” form to list individuals that match one or more of the following search fields: sampling unit, identity (search pattern with wildcards), alias (search pattern with wildcards), sex (male, female, unknown, or all), birth date after (date), birth date before (date), father identity (search pattern with wildcards), mother identity (search pattern with wildcards), and status (enabled or disabled). An individual's identity, alias, sex, birth date, father, mother, updating users, and update dates can be displayed. A list individuals form also can be used to view individuals' details, create individuals, update individuals, and delete individuals. A user can access an “import individuals” form to import individuals, including groupings and groups, from a file. Importing a file that contains both new and existing individuals can update existing individuals and create individuals.

A user can access a “list samples” form to list samples that match one or more of the following search fields: sampling unit, individual identity (search pattern with wildcards), sample name (search pattern with wildcards), sample tissue (search pattern with wildcards), and sample storage (search pattern with wildcards). Sample names, tissue type, manner of storage, updating users, and update dates can be displayed. A list samples form also can be used to view sample details, create samples, update samples, and delete samples. A user can access an “import samples” form to import samples from a file. Importing a file that contains both new and existing samples can update existing samples and create samples.

Phenotype administration forms: A user can access a “list phenotypes” form to display a list of phenotypes that match one or more of the following search fields: sampling unit, individual identity (choice of one or more), variable (choice of one or more). Individual identities, variables, values, updating users, and update dates can be displayed. A list phenotypes form also can be used to view phenotype details, create phenotypes, update phenotypes, and delete phenotypes. A user can access an “import phenotypes” form to import phenotypes from a file. In some configurations, three import modes can be accessed: “create new,” “update existing,” and “create or update.” The create new mode provides for the creation of new phenotypes, and old phenotypes are not allowed in the file. The update existing mode provides for the updating of old phenotypes, and new phenotypes are not allowed in the file. The create or update mode provides for the creation of new phenotypes and the updating of old phenotypes. A user can decide on an individual or collective basis whether particular phenotypes should be updated. A user can access a “phenotype status” form to display status information for phenotypes, including how many phenotypes are stored for a particular filter, variable set, or variable.

A user can access a “list variables” form to list variables that match one or more of the following search fields: sampling unit, name (search pattern with wildcards), type (choice of enumeration, number or both), and unit (search pattern with wildcards). Variable names, types, measurement units, associated comments, updating users, and update dates can be displayed. A list variables form also can be used to view variable details, create variables, update variables, and delete variables. A user can access an “import variables” form to import variables from a file.

A user can access a “list variable sets” form to list variable sets that match one or more of the following search fields: sampling unit, name (search pattern with wildcards), and variable (search pattern with wildcards). Variable set names, associated comments, updating users, and update dates can be displayed. A list variable sets form also can be used to view variable set details, create variable sets, update variable sets, and delete variable sets. A user can access a “variable set membership” form to add or delete variable set members. A user can access an “import variable sets” form to import variable sets from a file.

A user can access a “list U-variables” form to list unified variables that match one or more of the following search fields: name (search pattern with wildcards), type (choice of enumeration, number or both), and unit (search pattern with wildcards). Unified variable names, types, measurement units, associated comments, updating users, and update dates can be displayed. A list U-variables form also can be used to view unified variable details, create unified variables, update unified variables, and delete unified variables. A user can access a “map U-variables” form to map unified variables to variables in sampling units. A user can access an “import U-variables” form to import unified variables from a file. A user can access an “import U-variable mappings” form to import mappings from unified variables to variables.

A user can access a “list U-variable sets” form to list unified variable sets that match one or more of the following search fields: sampling unit, name (search pattern with wildcards), and unified variable (search pattern with wildcards). Unified variable set names, associated comments, updating users, and update dates can be displayed. A list U-variable sets form also can be used to view unified variable set details, create unified variable sets, update unified variable sets, and delete unified variable sets. A user can access a “U-variable set membership” form to add or delete unified variable set members. A user can access an “import U-variable sets” form to import unified variable sets from a file.

Genotype administration forms: A user can access a “list genotypes” form to list genotypes that match one or more of the following search fields: sampling unit, individual identity (choice of one or more); chromosome (choice of one or more), marker (choice of one or more), allele 1 (search pattern with wildcards), allele 2 (search pattern with wildcards), and reference (search pattern with wildcards). Individual identities, allele names, reference, security level, updating users, and date of last update can be displayed. A list genotypes form also can be used to view genotype details, create genotypes, update genotypes, and delete genotypes. A user can access an “update security level” form to update the security level attribute for a set of genotypes. Genotypes that match one or more of the following search fields define the genotype set: sampling unit, individual identity (choice of one or more), chromosome (choice of one or more), marker (choice of one or more), level (choice of one or more), user (choice of one or more), date after (date), and date before (date). A user can access an “import genotypes” form to import genotypes from a file. Three import modes can be accessed: “create new,” “update existing,” and “create or update.” The create new mode provides for the creation of new genotypes, and old genotypes are not allowed in the file. The update existing mode provides for the updating of old genotypes, and new genotypes are not allowed in the file. The create or update mode provides for the creation of new genotypes and the updating of old genotypes. In modes where existing genotypes are updated, a list of genotypes to be updated can be displayed. A user can decide on an individual, or collective basis whether particular genotypes should be updated. A user can access a “genotype status ” form to display status information regarding genotypes, including how many genotypes are stored for a particular filter, marker set, or marker.

A user can access a “list markers” to list markers that match one or more of the following search fields: sampling unit and chromosome (choice of one or more). Marker names, associated comments, chromosome on which a marker is located, updating users, and update dates can be displayed. A list markers variables form also can be used to view marker and allele details, create markers, update markers, delete markers, create alleles, update alleles, and delete alleles. A user can access an “import markers” form to import markers, including alleles from a file. A user can access an “import library markers” form to import library markers, including library alleles, from a library (i.e., a set of library markers).

A user can access a “list marker sets” form to list marker sets that match one or more of the following search fields: sampling unit, name (search pattern with wildcards), comment (search pattern with wildcards), and marker (search pattern with wildcards). Marker set names, associated comments, updating users, and update dates can be displayed. A list marker sets form also can be used to view marker set details, create marker sets, update marker sets, and delete marker sets. A user can access a “marker set membership” form to add or delete marker set members. A user can access a “marker set positions” form to view and edit the genetic positions for markers in a marker set. A user can access an “import marker sets” form to import marker sets, including positions, from a file.

A user can access a “list U-markers” form to list unified markers that are linked to one or more chromosomes. U-marker set names, associated comments, updating users, and update dates can be displayed. A list U-markers form also can be used to view unified variable sets, including unified alleles, create unified variable sets, update unified variable sets, delete unified variable sets, view details for unified alleles, create unified alleles, update unified alleles, and delete unified alleles. A user can access a “map U-markers” form to map unified markers to markers in sampling units, and to map alleles to unified alleles. A user can access an “import U-markers” form to import unified markers from a file. A user can access an “import U-marker mappings” form to import mappings from unified markers to markers, and to import alleles to unified alleles.

A user can access a “list U-marker sets” form to list unified marker sets that match one or more of the following search fields: name (search pattern with wildcards), comment (search pattern with wildcards), and unified variable (search pattern with wildcards). U-marker set names, associated comments, updating users; and update dates can be displayed. A list U-marker sets form also can be used to view unified marker set details, create unified marker sets, update unified marker sets, and delete unified marker sets. A user can access a “U-marker set membership” form to add or delete unified marker set members. A user can access a “U-marker set positions” form to view and edit the genetic positions for unified markers in unified marker sets. A user can access an “import U-marker sets” form to import unified marker sets from a file.

Analyses administration forms: A user can access a “list filters” form to list filters that match one or more of the following search fields: name (search pattern with wildcards) and expression (search pattern with wildcards). Filter names, expressions, updating users, and update dates can be displayed. A list filters form also can be used to view filter details, create filters, edit filters, test filters, and delete filters.

A user can access a “start file generation” form to create a file generation, including data files. Two modes of file generation can be accessed, “single mode” and “multiple mode.” Single mode file generation provides for the analysis of one sampling unit, and a user specifies the sampling unit, filter, marker set, variable set, and type of analysis. Multiple mode operation provides for the analysis of several sampling units, and a user specifies the sampling unit set, filter for each sampling unit, unified marker set, unified variable set, and type of analysis. File generation can include, for example, general tables, and linkage maps. A variety of linkage maps can be created by those of skill in the art, using for example Crimap, Makeped, or Mapmaker software. See e.g., Green, P., Falls K., and Crook, S. (1990) Documentation for CRI-MAP, version 2.4. Washington University School of Medicine, St Louise, Mo.; Lander et al. (1987) Mapmaker, an interactive computer package for constructing primary genetic linkage maps of experimental and natural populations. Genomics 1:174-181; Lincoln et al. (1992) Constructing genetic maps with Mapmaker/Exp 3.0. Whitehead Institute Technical Report 3rd Ed.; Lincoln et al. (1992) Mapping genes controlling quantitative traits with Mapmaker/QTL 1.1, Whitehead Institute Technical Report 2nd Ed.; and Lathrop et al (1984) Strategies for multilocus linkage analysis in humans. Proc Natl Acad Sci U.S.A. 81:3443-6.

A user can access a “list file generations” form to list file generations that match one or more of the following search fields: name (search pattern with wildcards), mode (choice of single, multiple or both), type (choice of one or more), and status (choice of generated, being generated, error, or all). File generation names, mode, type, status, size, updating users, and update dates can be displayed. A list file generations also can be used to view analysis details, view download result details, update file generations, and delete file generations.

The information related to the forms described above may be presented to a user an any number of combinations, for example, as printed reports or as reports viewed on a computer monitor. The information may also be compiled, combined or translated to form tables, graphs or other like entities for interpreting the data.

### Research System Output

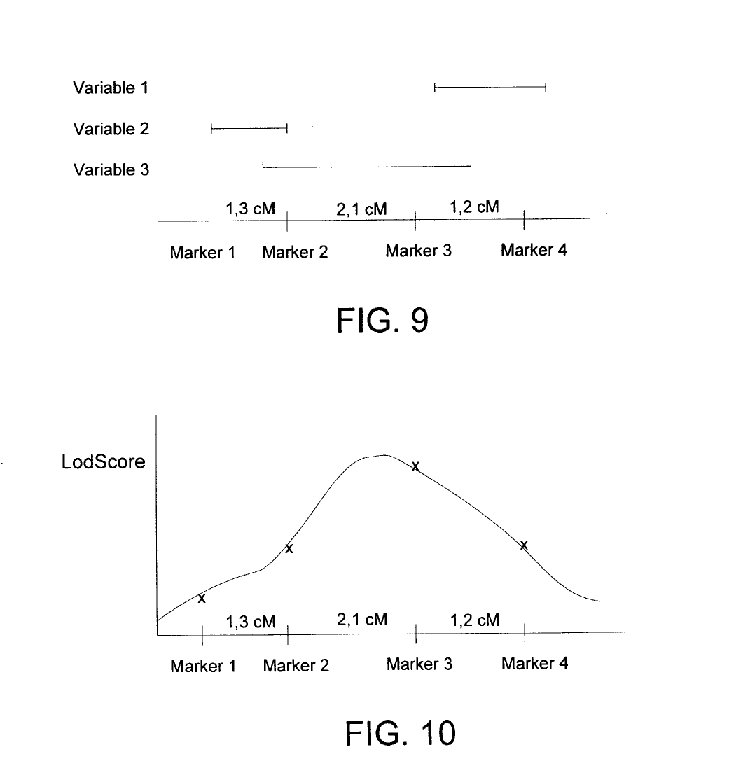

As described above, genetic research system 8 provides flexible information storage, processing, and analysis structures that can facilitate collaboration between genetic researchers. Researchers interact with genetic research system 8 and invoke data analysis modules 28 to process the genetic data stored within database system 22. In one configuration, genetic research system 8 communicates output to computer 10 for display to a user. FIGS. 9 and 10 illustrate two exemplary output charts produced by a genetic research system 8 upon processing genetic research data. FIG. 9 is a genetic map that shows the genetic distance between a set of markers within a Marker set object 74, their relative order on a chromosome within Chromosome object 41, and confidence intervals for three variables. FIG. 10 shows linkage values (lod scores) for a variable within Variable object 60 over the set of markers. Other output is readily produced by data analysis modules 28 executing other specialized algorithms.

### Operating Environment for Research Computer or Server

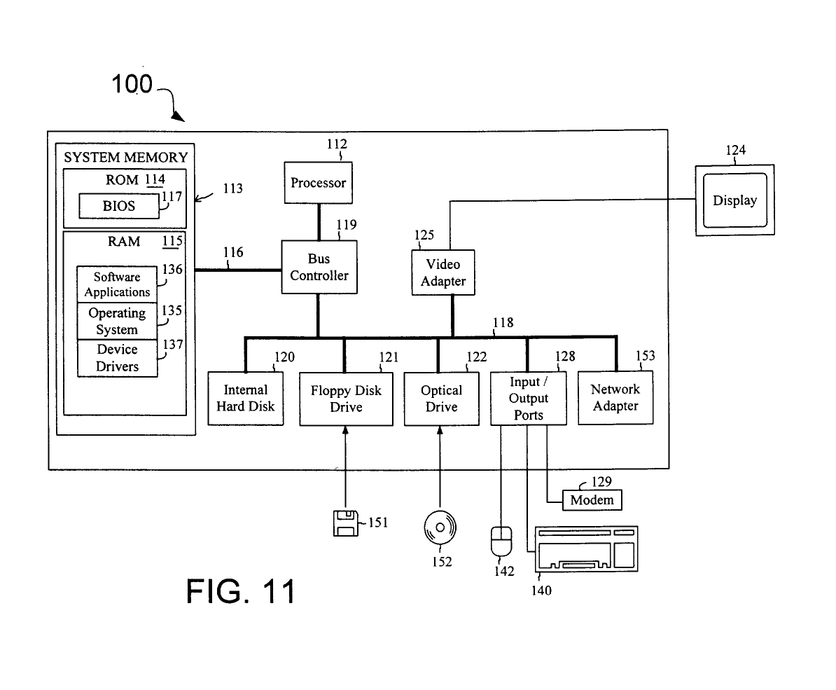

FIG. 11 shows a computer system 100 that a researcher in a genetic research environment can use to interact with genetic research system 8. Computer system 100 can provide an operating environment suitable for use as a research computer 10, as well as a server within genetic research system 8. In various configurations, computer system 100 represents any server, personal computer, laptop or even a battery-powered, pocket-sized, mobile computer known as a hand-held PC or personal digital assistant (PDA).

Computer system 100 includes a processor 112 that in one embodiment belongs to the PENTIUM® family of microprocessors manufactured by the Intel Corporation of Santa Clara, Calif. The invention also can be implemented on computers based upon other microprocessors, such as the MIPS® family of microprocessors from the Silicon Graphics Corporation, the POWERPC® family of microprocessors from both the Motorola Corporation and the IBM Corporation, the PRECISION ARCHITECTURE® family of microprocessors from the Hewlett-Packard Company, the SPARC® family of microprocessors from the Sun Microsystems Corporation, or the ALPHA® family of microprocessors from the Compaq Computer Corporation. Computer system 100 also includes system memory 113, including read only memory (ROM) 114 and random access memory (RAM) 115, which is connected to a processor 112 by a system data/address bus 116. ROM 114 represents any device that is primarily read-only including electrically erasable programmable read-only memory (EEPROM), flash memory, etc. RAM 115 represents any random access memory such as Synchronous Dynamic Random Access Memory. Computer system 100 also can include a modem 129, which can be internal or external to a system 100. Modem 129 typically is used to communicate over wide area networks (not shown), such as the global Internet using either a wired or wireless connection.

Within computer system 100, an input/output bus (bus) 118 is connected to a data/address bus 116 via a bus controller 119. In one embodiment, input/output bus 118 is implemented as a standard Peripheral Component Interconnect (PCI) bus. Bus controller 119 examines all signals from processor 112 to route the signals to the appropriate bus. Signals between processor 112 and system memory 113 are passed through bus controller 119. Signals from processor 112 intended for devices other than system memory 113 are routed onto input/output bus 118. Various devices can be connected to bus 118, including a hard disk drive 120, a floppy drive 121 that is used to read a floppy disk 151, and an optical drive (e.g., a CD-ROM drive) 122, that is used to read an optical disk 152. A video display 124 or other kind of display device can be connected to bus 118 via a video adapter 125. Users provide commands and information into computer system 100 by using a keyboard 140 and/or a pointing device, (e.g. a mouse) 142, which are connected to bus 118 via input/output ports 128. Other types of pointing devices include track pads, track balls, joysticks, data gloves, head trackers, and other devices suitable for positioning a cursor on video display 124.

Software applications 136 and data typically are stored via a memory storage devices, which may include hard disk 120, floppy disk 151, and CD-ROM 152, and are copied to RAM 115 for execution. In one embodiment, software applications 136 are stored in ROM 114 and are copied to RAM 115 for execution or are executed directly from ROM 114. In general, an operating system 135 executes software applications 136 and carries out instructions issued by a user. For example, when a user wants to load software application 136, operating system 135 interprets the instruction and causes processor 112 to load software application 136 into RAM 115 from either hard disk 120 or optical disk 152. Once software application 136 is loaded into RAM 115, it can be executed by processor 112. In case of large software applications 136, processor 112 can load various portions of program modules into RAM 115 as needed.

The Basic Input/Output System (BIOS) 117 for computer system 100 is a set of basic executable routines that have conventionally helped to transfer information between the computing resources within computer system 100. Operating system 135 or other software applications 136 use these low-level service routines. In one embodiment, computer system 100 includes a registry database (not shown) that holds configuration information for computer system 100. For example, the Windows® operating system by Microsoft Corporation of Redmond, Wash., maintains the registry in two hidden files, called USER.DAT and SYSTEM.DAT, located on a permanent storage device such as an internal disk.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetic research system comprising:
a) one or more genotype data structures to store genotype data obtained from individuals belonging to a plurality of sampling units;
b) one or more phenotype data structures to store phenotype data obtained from individuals belonging to a plurality of sampling units;
c) a project data structure to store information about genetic research projects that include one or more of the sampling units;
d) a species data structure to store information about biological species included in the genetic research projects;
e) a chromosome data structure to store information about the chromosomes of the biological species; and
f) a front-end gateway that provides access to information derived from the genotype and phenotype data.

2. The system of claim 1, further comprising:
a) a role data structure to store information about roles that users may be assigned in the projects;
b) a privilege data structure to store information about the operations that the users can perform using the system; and
c) a user data structure to store information about the users.

3. The system of claim 1, further comprising a sampling unit data structure to store information about the sampling units.

4. The system of claim 1, further comprising an individual data structure to store information about the individuals.

5. The system of claim 4, further comprising a sample data structure to store information about samples obtained from the individuals.

6. The system of claim 4, further comprising a grouping data structure to store information about genetically relevant groupings to which the individuals can belong.

7. The system of claim 6, further comprising a group data structure to store information about genetically relevant groups within the groupings.

8. The system of claim 1, further comprising:
a) a variable data structure to store information about phenotypic traits measured or observed for individuals in the sampling groups; and
b) a variable set data structure to store information about the variables that are to be used when generating data files.

9. The system of claim 1, further comprising:
a) a marker data structure to store information about genetic markers examined for individuals in the sampling groups;
b) an allele data structure to store information about alleles of one or more of the genetic markers;
c) a marker set data structure to store information about the genetic markers that are to used when generating data files.

10. The system of claim 9 further comprising a position data structure to store information about the genetic position of the markers.

11. A genetic research system comprising:
a) one or more genotype data structures to store genotype data obtained from individuals belonging to a plurality of sampling units;
b) one or more phenotype data structures to store phenotype data obtained from individuals belonging to a plurality of sampling units;
c) one or more genotype proxy data structures that permit the collective analysis of at least some of the genotype data while maintaining the genotype data pertaining to individual sampling units in the genotype data structures;
d) one or more phenotype proxy data structures that permit the collective analysis of at least some of the phenotype data while maintaining the phenotype data pertaining to individual sampling units in the phenotype data structures; and
e) a front-end gateway that provides access to information derived from the genotype and phenotype data.

12. The system of claim 11, further comprising a project data structure to store information about genetic research projects that include one or more of the sampling units.

13. The system of claim 12, further comprising:
a) a role data structure to store information about roles that users may be assigned in the projects;
b) a privilege data structure to store information about the operations that the users can perform using the system; and
c) a user data structure to store information about the users.

14. The system of claim 12, further comprising a sampling unit data structure to store information about the sampling units.

15. The system of claim 12, further comprising an individual data structure to store information about the individuals.

16. The system of claim 15, further comprising a sample data structure to store information about samples obtained from the individuals.

17. The system of claim 15, further comprising a grouping data structure to store information about genetically relevant groupings to which the individuals can belong.

18. The system of claim 17, further comprising a group data structure to store information about genetically relevant groups within the groupings.

19. The system of claim 11, further comprising:
a) a variable data structure to store information about the phenotypic traits measured or observed for individuals in the sampling groups; and
b) a variable set data structure to store information about the variables that are to be used when generating data files.

20. The system of claim 19, wherein the phenotype proxy data structures comprise:
a) a unified variable data structure to store information about unified variables that refer to and associate variables pertaining to different sampling groups; and
b) a unified variable set data structure to store information about the unified variables that are to be used when generating data files.

21. The system of claim 11, further comprising:
a) a marker data structure to store information about genetic markers examined for individuals in the sampling groups;
b) an allele data structure to store information about alleles of one or more of the genetic markers;
c) a marker set data structure to store information about the genetic markers that are to used when generating data files.

22. The system of claim 21, further comprising a position data structure to store information about the genetic position of the markers.

23. The system of claim 21, wherein the genotype proxy data structures comprises:
a) a unified marker data structure to store information about unified markers that refer to and associate markers pertaining to different sampling groups; and
b) a unified allele data structure to store information about unified alleles that refer to and associate alleles pertaining to different sampling groups; and
c) a unified marker set data structure to store information about the unified markers that are to be used when generating data files.

24. The system of claim 23, further comprising a unified position data structure to store information about unified positions that refer to and associate positions pertaining to different sampling groups.

25. A method for providing access to a genetic research system, comprising:
a) receiving a request from a user to access a genotype data structure within the system, wherein the genotype data structure includes nucleic acid sequence data and a level attribute;
b) querying a project data object within the system to determine which entries within the genetic research objects the user can access;
c) querying a role data structure and a privileges data structure within the system to determine a set of operations that the user is allowed to perform; and
d) providing access to the system based on the results of the queries.

26. A method for providing genetic research information to a user, comprising:
a) providing the user access to a genetic research system including one or more genotype data structures to store genotype data obtained from individuals belonging to a plurality of sampling units, and one or more phenotype data structures to store phenotype data obtained from individuals belonging to a plurality of sampling units;
b) using one or more genotype proxy data structures to associate genotype data for individuals in different sampling units while maintaining genotype data for individual sampling units in the genotype data structures;
c) using one or more phenotype proxy data structures to associate phenotype data for individuals in different sampling units while maintaining phenotype data for individual sampling units in the phenotype data structures; and
d) providing the user with information derived from the associated phenotype data and the associated genotype data.
